(19) United States
(12) Patent Application Publication
(10) Pub. No.: US 2024/0374438 A1
(43) Pub. Date: Nov. 14, 2024

(54) SUBDIVIDABLE DENTAL ABSORBENT BLOCK

(71) Applicant: Gary Houseman, Saskatoon (CA)

(72) Inventor: Gary Houseman, Saskatoon (CA)

(21) Appl. No.: 18/210,441

(22) Filed: Jun. 15, 2023

(30) Foreign Application Priority Data
May 12, 2023 (CA) 3199490

(51) Int. Cl. A61F 13/20; A61C 19/06

(52) U.S. Cl. CPC A61F 13/2008; A61C 19/063; A61F 13/2034; A61F 13/2077

(57) ABSTRACT

An absorbent block of a length greater than a single tooth extraction site, the block subdividable into selected customized widths for application to a dental extraction site. The block may have a longitudinal notch in the surface pressed against the extraction site, or two opposed notches where corresponding upper and lower teeth are extracted. Pre-cut lines or surface marked lines indicate available widths for block subdivisions. The block may be curved as appropriate to align with partial or full arch teeth extraction procedures.

### TECHNICAL FIELD OF THE INVENTION

The present invention relates to absorbent products for surgical procedures, and more particularly to absorbent products for use by patients after dental extraction procedures.

### BACKGROUND OF THE INVENTION

It is known in the art of dental procedures including oral surgery that post-procedure bleeding requires tending until natural coagulation can occur (with or without application of coagulant compositions to aid the natural ability). As dental patients normally leave the surgical location after the procedure is complete, such post-procedure tending is usually undertaken by patients themselves.

One product in ubiquitous use for such self-tending is a gauze pad. Such gauze pads are commonly supplied by the dentist or surgeon office to the patient, which gauze pads are thin sheets to be folded over numerous times to form a wad which is then inserted against the extraction site, the patient then biting down on the wad and thus slowing the bleeding until coagulation is concluded. However, it is known that this common solution can result in poor and/or delayed coagulation due, for example, to inadequate wad depth and/or poor coverage of the extraction site.

Various prior art solutions have been proposed to address this need. For example, U.S. Pat. No. 3,705,585 to Saffro discloses a preformed surgical sponge for oral surgery that is configured to press against the extraction site and wrap partially around the front and back gums adjacent the extraction site, including embodiments where upper and lower teeth are removed. In a further example, U.S. Pat. No. 4,372,314 to Wall discloses a dental sponge formable in various shapes including curved to mate with a dental arch. In a further example, U.S. Pat. No. 8,809,614 to Wilhoit discloses a dental wound dressing comprising a preformed gauze block configured for deformation against upper and/or lower extraction sites.

However, the prior art solutions imply consistent extraction site widths and dental arch sizes, such that use by patients of such prior art solutions might result in discomfort or inutility rising from using a pad of incorrect size.

What is needed, therefore, is a dental product that provides absorbent functionality while being customizable to different arch and extraction site sizes.

### SUMMARY OF THE INVENTION

According to a first broad aspect of the present invention, there is provided an absorbent block for use in post-extraction dental care, the absorbent block comprising:
a length sufficient to cover at least two adjacent dental extraction sites on a dental arch;
the absorbent block subdividable into a plurality of portions of defined widths;
one or more of the portions selectable to cover a target dental extraction site; and
the one or more of the portions configured for removal together from the absorbent block as a block division of selected total width and for application to and pressuring against the target dental extraction site.

In some exemplary embodiments, the absorbent block comprises absorbent material in a gauze exterior cover. The absorbent material is preferably stacked gauze. The stacked gauze may comprise layers of gauze adhered together, or alternatively the stacked gauze may comprise layers of gauze interwoven together.

In some exemplary embodiments, the absorbent block further comprises a first longitudinal notch on one surface of the absorbent block along the length of the absorbent block, the first longitudinal notch sized and configured for wrapping over the target dental extraction site. Where corresponding upper and lower teeth are extracted and the target dental extraction site comprises upper and lower target dental extraction sites, exemplary embodiments may further comprise a second longitudinal notch on an opposite surface of the absorbent block along the length of the absorbent block, the second longitudinal notch sized and configured for wrapping over the upper target dental extraction site and the first longitudinal notch is sized and configured for wrapping over the lower target dental extraction site.

The absorbent block is preferably subdividable at lines pre-cut into the absorbent block but that extend only through one or more outer surfaces of the absorbent block, the lines indicating the defined widths. Alternatively, the absorbent block may be subdividable at lines visually marked on an outer surface of the absorbent block, without pre-cutting the block surface, the lines indicating the defined widths. The defined widths preferably comprise varying defined widths allowing customization of the block division width.

In some exemplary embodiments, the absorbent block is curved along at least part of the length and the length is sufficient to cover the target dental extraction site for extraction of a plurality of teeth along a full arch. In such cases, a first curved longitudinal notch may be provided on one surface of the absorbent block along the length of the absorbent block, the first curved longitudinal notch sized and configured for wrapping over the target dental extraction site. Where the plurality of teeth are extracted from corresponding upper and lower arches and the target dental extraction site therefore comprises upper and lower target dental extraction sites, the exemplary embodiments may comprise a second curved longitudinal notch on an opposite surface of the absorbent block along the length of the absorbent block, the second curved longitudinal notch sized and configured for wrapping over the upper target dental extraction site and the first curved longitudinal notch is sized and configured for wrapping over the lower target dental extraction site. As the block is divided into selectable portions, in some exemplary embodiments such curved blocks comprise end portions that are configured for removal to form a full arch block having a length equivalent to a length of the full arch itself.

In some exemplary embodiments, the absorbent block further comprises an application surface configured for receipt of a coagulant for pressing against the target dental extraction site. Where the block comprises a longitudinal notch, the longitudinal notch is preferably configured for receipt of a coagulant for pressing against the target dental extraction site.

A detailed description of exemplary embodiments of the present invention is given in the following. It is to be understood, however, that the invention is not to be construed as being limited to these embodiments. The exemplary embodiments are directed to particular applications of the present invention, while it will be clear to those skilled in the art that the present invention has applicability beyond the exemplary embodiments set forth herein.

### BRIEF DESCRIPTION OF THE DRAWINGS

In the accompanying drawings, which illustrate exemplary embodiments of the present invention:

FIG. 1a is a side perspective view of an exemplary straight absorbent block with surface markings and a single notch.

FIG. 1b is a top plan view of the block of FIG. 1a.

FIG. 1c is a bottom perspective view of the block of FIG. 1a.

FIG. 2a is a top perspective view of different-sized exemplary block divisions.

FIG. 2b is a top plan view of the block divisions of FIG. 2a illustrating the different widths.

FIG. 3a is a side perspective view of an exemplary block division sized for a single-tooth extraction applied to the extraction site.

FIG. 3b is a side perspective view of an exemplary block division for a double-tooth extraction applied to the extraction site.

FIG. 3c is a side perspective view of an exemplary block division for a triple-tooth extraction applied to the extraction site.

FIG. 4a is top and bottom perspective views of an exemplary single-notch curved block for full arch coverage.

FIG. 4b is a front perspective view of the block of FIG. 4a applied to an extraction site.

FIG. 5a is a rear elevation view of an exemplary double-notch curved block for double arch coverage.

FIG. 5b is a front perspective view of the block of FIG. 5a applied to a lower extraction site and showing the upper notch.

Exemplary embodiments will now be described with reference to the accompanying drawings.

### DETAILED DESCRIPTION OF EXEMPLARY EMBODIMENTS

Throughout the following description, specific details are set forth in order to provide a more thorough understanding to persons skilled in the art. However, well known elements may not have been shown or described in detail to avoid unnecessarily obscuring the disclosure. The following description of examples of the invention is not intended to be exhaustive or to limit the invention to the precise form of any exemplary embodiment. Accordingly, the description and drawings are to be regarded in an illustrative, rather than a restrictive, sense.

The present invention is directed to an absorbent block that is subdividable into selected block divisions of a desired width. In this way, a user may generate customized block divisions sized appropriately for a specific dental extraction, from single teeth to full or even double arches. By allowing a user to selectively generate custom-sized absorbent block divisions, the coagulation may be further supported. As noted above, the block may be composed of any appropriate absorbent material, such as stacked gauze, and is preferably but not necessarily covered by an outer gauze cover.

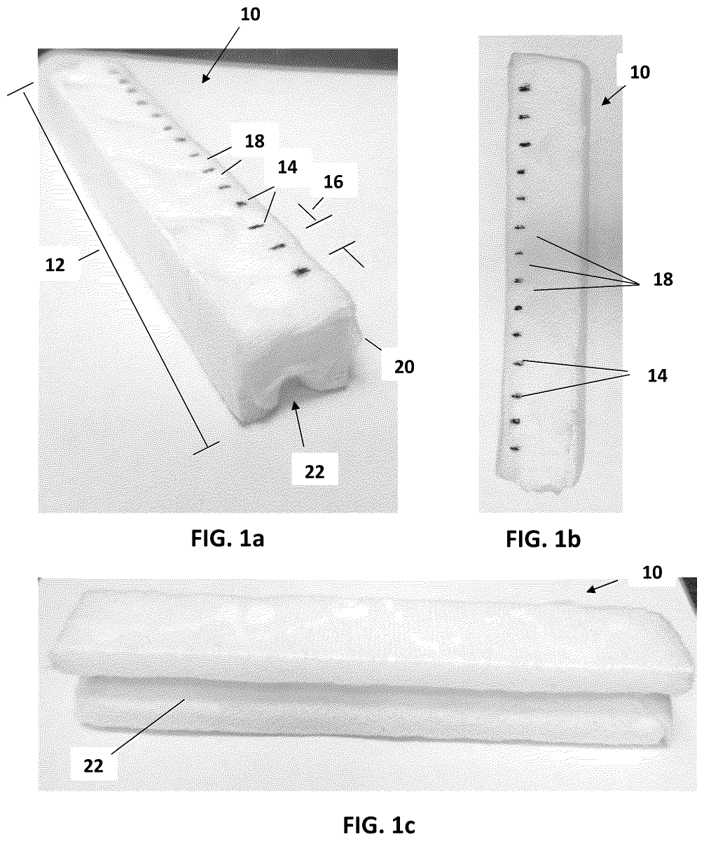

Turning now to FIGS. 1a to 1c, a first exemplary embodiment is illustrated. An absorbent block 10 is shown, of a length 12 sufficient to be subdivided into various desired custom portions. The block 10 is provided with markings 14 which can be used by a user to select a desired total length appropriate for the width of a specific extraction site. The markings 14 are shown dividing the block 10 into equal parts of spacing 16 (which would be the widths of the block divisions when divided from the block 10), but the markings 14 could be given varying spacing. Further, although not shown, instead of or in addition to the markings 14 the block 10 could be provided with pre-cuts at the markings 14 to aid in division of the block 10. The block 10 is provided with an outer gauze cover 20.

The markings 14 divide the block 10 into portions 18. Each such portion 18 may be selected as a block division for use with an extraction site, or a series of adjacent portions 18 may be selected and removed together from the block 10 to form a larger width to match the target extraction site.

As can be seen in FIGS. 1a and 1c, the block 10 comprises a longitudinal notch 22 running the length 12 of the block 10. The notch 22 is configured for contacting and partially enclosing the extraction site when applied. The surface of the notch 22 may be further provided with an appropriate coagulant composition in liquid or gel form known to those in the art.

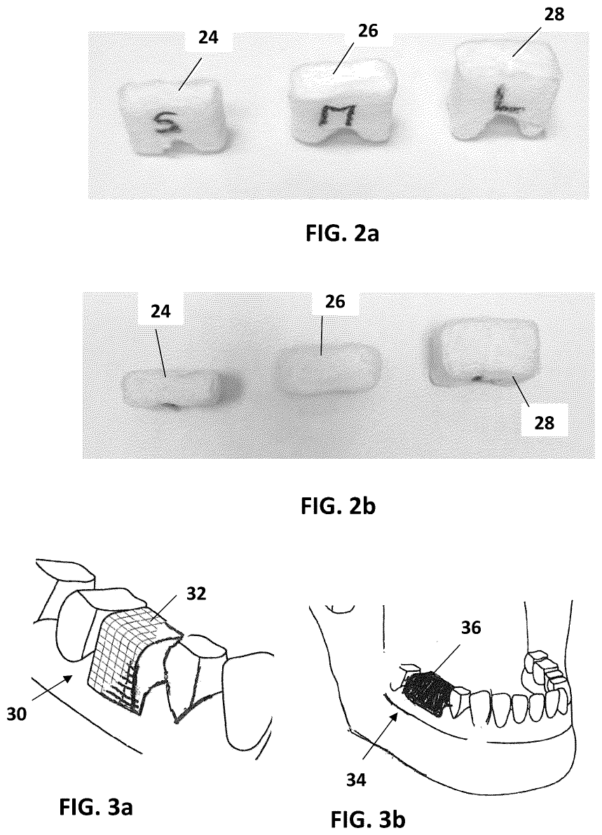

Turning now to FIGS. 2a and 2b, exemplary block divisions are shown. As noted above, a user would cut or otherwise divide the block 10 into customized portions of desired length to match the width of a target extraction site, and the block 10 may be provided with markings or pre-cuts to aid the user in this. As extraction sites, and the dental arches where they are located, can vary significantly in size, being able to generate customized block divisions of desired length is of utility. For example, a small block division 24, a medium block division 26 and a large block division 28 are illustrated, or varying lengths (and thus of varying block division widths to mate with a specific extraction site). Based on a specific target extraction site, the user could select which block division size would be most appropriate for the extraction site and divide the block 10 accordingly.

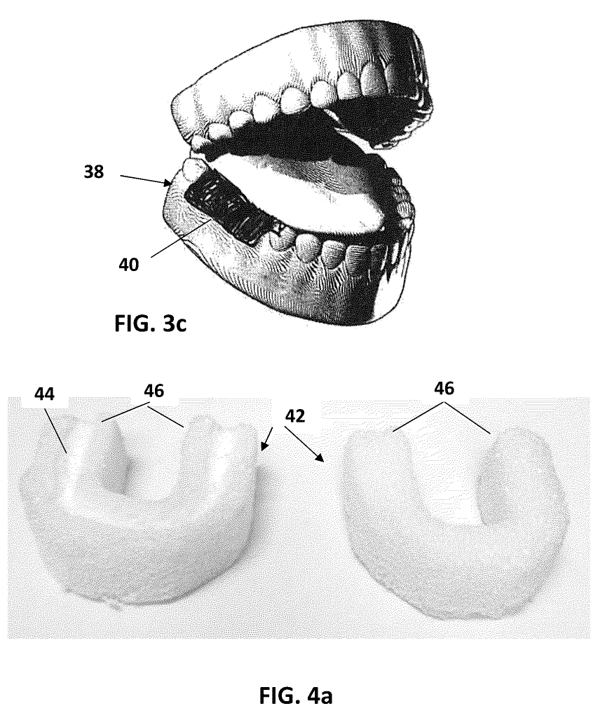

Turning to FIGS. 3a to 3c, this utility is further illustrated for extractions involving one or more teeth. FIG. 3a illustrates a situation where a single lower tooth has been extracted from an extraction site 30, and a block division 32 of suitable size can be removed from an absorbent block as described above to fit the specific gap width. FIG. 3b illustrates a situation where two adjacent lower teeth have been extracted from an extraction site 34, and a block division 36 of suitable size for a two-tooth gap can be removed from an absorbent block as described above to fit the specific gap width. FIG. 3c illustrates a situation where three adjacent lower teeth have been extracted from an extraction site 38, and a block division 40 of suitable size for a three-tooth gap can be removed from an absorbent block as described above to fit the specific gap width. In this way, the user may create various customized block division lengths matching the gap width of the specific extraction site.

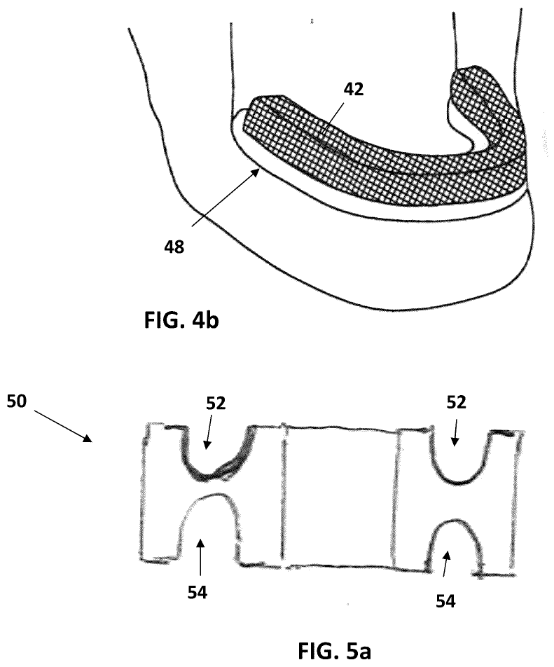

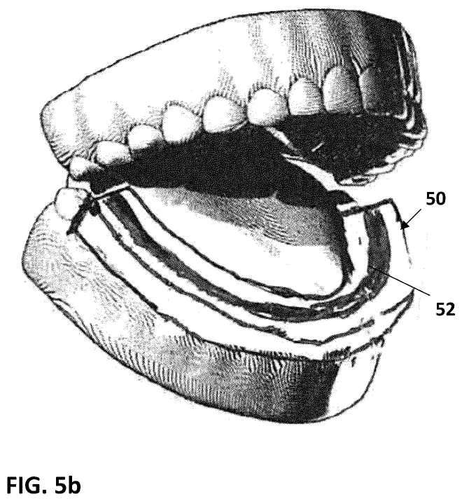

It is also known in the art to extract all teeth from one or both the upper and lower dental arches. FIGS. 4a to 5b illustrate embodiments addressing such situations.

FIGS. 4a to 4b illustrate a single full arch extraction scenario. In this case, a curved absorbent block 42 may be used to match the curvature of the dental arch. FIG. 4a illustrates both sides of the block 42, with one of the sides provided with a curved notch 44. Further, the block 42 may have end portions 46 removed as described above to ensure that the total length of the block 42 matches the length of the dental arch. FIG. 4b illustrates the block 42 applied to a lower full arch extraction site 48.

Turning now to FIGS. 5a and 5b, a double full arch extraction scenario is illustrated. In this scenario, the teeth of both the upper and lower arches have been extracted, and an absorbent block 50 is prepared. The block 50 is again curved to match the shape of the upper and lower arches, and in this embodiment includes both upper and lower notches 52, 54 to mate with the extraction sites. FIG. 5b illustrates the block 50 pressed downwardly against the lower arch extraction site, with the upper notch 52 shown for illustration purposes—the patient would bite down on this upper notch 52 so that both the upper and lower arches engaged the block 50.

As can be seen, the use of straight or curved absorbent blocks, with guiding markings and/or pre-cuts at equal or varying distances can provide a dentist and/or patient with a customizable absorbent material solution that can be created to match the size of any extraction site. Unlike prior art solutions which teach set sizes and shapes of absorbent pads, embodiments according to the present invention may provide more comfortable and better-fitted products to aid in bleeding control and coagulation.

The foregoing is considered as illustrative only of the principles of the present invention. The scope of the claims should not be limited by the exemplary embodiments set forth in the foregoing, but should be given the broadest interpretation consistent with the specification as a whole.

1. An absorbent block for use in post-extraction dental care, the absorbent block comprising:
a length sufficient to cover at least two adjacent dental extraction sites on a dental arch;
the absorbent block subdividable into a plurality of portions of defined widths;
one or more of the portions selectable to cover a target dental extraction site; and
the one or more of the portions configured for removal together from the absorbent block as a block division of selected total width and for application to and pressuring against the target dental extraction site.

2. The absorbent block of claim 1, wherein the absorbent block comprises absorbent material in a gauze exterior cover.

3. The absorbent block of claim 2, wherein the absorbent material is stacked gauze.

4. The absorbent block of claim 1, and further comprising a first longitudinal notch on one surface of the absorbent block along the length of the absorbent block, the first longitudinal notch sized and configured for wrapping over the target dental extraction site.

5. The absorbent block of claim 4, and further comprising, where corresponding upper and lower teeth are extracted and the target dental extraction site comprises upper and lower target dental extraction sites, a second longitudinal notch on an opposite surface of the absorbent block along the length of the absorbent block, the second longitudinal notch sized and configured for wrapping over the upper target dental extraction site and the first longitudinal notch is sized and configured for wrapping over the lower target dental extraction site.

6. The absorbent block of claim 1, wherein the absorbent block is subdividable at lines precut into the absorbent block but that extend only through one or more outer surfaces of the absorbent block, the lines indicating the defined widths.

7. The absorbent block of claim 1, wherein the absorbent block is subdividable at lines visually marked on an outer surface of the absorbent block, the lines indicating the defined widths.

8. The absorbent block of claim 1, wherein the defined widths comprise varying defined widths allowing customization of the block division width.

9. The absorbent block of claim 1, wherein the absorbent block is curved along at least part of the length and the length is sufficient to cover the target dental extraction site for extraction of a plurality of teeth along a full arch.

10. The absorbent block of claim 9, and further comprising a first curved longitudinal notch on one surface of the absorbent block along the length of the absorbent block, the first curved longitudinal notch sized and configured for wrapping over the target dental extraction site.

11. The absorbent block of claim 10, and further comprising, where the plurality of teeth are extracted from corresponding upper and lower arches and the target dental extraction site therefore comprises upper and lower target dental extraction sites, a second curved longitudinal notch on an opposite surface of the absorbent block along the length of the absorbent block, the second curved longitudinal notch sized and configured for wrapping over the upper target dental extraction site and the first curved longitudinal notch is sized and configured for wrapping over the lower target dental extraction site.

12. The absorbent block of claim 3, wherein the stacked gauze comprises layers of gauze adhered together.

13. The absorbent block of claim 3, wherein the stacked gauze comprises layers of gauze interwoven together.

14. The absorbent block of claim 1, and further comprising an application surface configured for receipt of a coagulant for pressing against the target dental extraction site.

15. The absorbent block of claim 4, wherein the longitudinal notch is configured for receipt of a coagulant for pressing against the target dental extraction site.

16. The absorbent block of claim 9, wherein end portions of the absorbent block are configured for removal to form a full arch block having a length equivalent to a length of the full arch.
